(19) United States
(12) Patent Application Publication
(10) Pub. No.: US 2022/0313998 A1
(43) Pub. Date: Oct. 6, 2022

(54) AUDITORY PROSTHETIC DEVICES USING EARLY AUDITORY POTENTIALS AS A MICROPHONE AND RELATED METHODS

(71) Applicants: Ohio State Innovation Foundation, Columbus, OH (US); The University of North Carolina at Chapel Hill, Chapel Hill, NC (US); Washington University, St. Louis, MO (US)

(72) Inventors: Oliver F. ADUNKA, Columbus, OH (US); Douglas C. FITZPATRICK, Chapel Hill, NC (US); Craig A. BUCHMAN, St. Louis, MO (US)

(21) Appl. No.: 17/610,578

(22) PCT Filed: Jul. 9, 2020
(86) PCT No.: PCT/US2020/041369

§ 371 Date: Nov. 11, 2021

Related U.S. Application Data
(60) Provisional application No. 62/872,466, filed on Jul. 10, 2019.

(51) Int. Cl. A61N 1/36; A61N 1/05

(52) U.S. Cl. CPC A61N 1/36038; A61N 1/0541

(57) ABSTRACT

Described herein are devices and methods that use the electrical potentials that arise naturally in the cochlea through the activity of sensory cells and auditory neurons and resemble the output from a microphone. An example auditory prosthetic device includes an electrode array that is configured for insertion into at least a portion of a subject's cochlea and a receiver-stimulator operably coupled to the electrode array. The electrode array is configured for electrical recording and stimulation.

### CROSS-REFERENCE TO RELATED APPLICATIONS

This application claims the benefit of U.S. provisional patent application No. 62/872,466, filed on Jul. 10, 2019, and entitled “AUDITORY PROSTHETIC DEVICES USING EARLY AUDITORY POTENTIALS AS A MICROPHONE AND RELATED METHODS,” the disclosure of which is expressly incorporated herein by reference in its entirety.

### BACKGROUND

Current semi-implantable hearing devices including modern cochlear implants use an external microphone to record sound and deliver a processed signal to the internal components. A design with benefits of comfort and potentially performance would be to use a ‘fully implantable’ microphone with no external components. Current efforts to design such microphones involve various types of pressure and vibration sensors placed either under the skin or in the middle or inner ear.

### SUMMARY

Described herein are devices and methods that use the electrical potentials that arise naturally in the cochlea through the activity of sensory cells and auditory neurons and resemble the output from a microphone.

An example auditory prosthetic device is described herein. The example auditory prosthetic device includes an electrode array that is configured for insertion into at least a portion of a subject's cochlea and a receiver-stimulator operably coupled to the electrode array. The electrode array is configured for electrical recording and stimulation.

In some implementations, the receiver-stimulator is configured to receive an early auditory potential recorded by the electrode array, process the early auditory potential to generate a stimulation signal, and transmit the stimulation signal to the electrode array. The early auditory potential is recorded using the electrode array, and the early auditory potential includes cochlear microphonic.

Alternatively or additionally, the stimulation signal is applied within the subject's cochlea using the electrode array.

Alternatively or additionally, the receiver-stimulator includes a digital signal processor (DSP), and the DSP is configured to process the early auditory potential to generate the stimulation signal. Optionally, processing the early auditory potential to generate the stimulation signal includes detecting and removing a stimulus artifact. In some implementations, the stimulus artifact is detected and removed using at least one of a template matching technique, a linear interpolation technique, or low pass filtering.

Alternatively or additionally, the electrode array includes a plurality of electrodes. Optionally, the early auditory potential is recorded at one or more of the electrodes of the electrode array. Alternatively or additionally, the early auditory potential is recorded at each of the electrodes of the electrode array. Optionally, the electrodes of the electrode array are arranged to correspond to different tonotopic locations of the subject's cochlea.

Alternatively or additionally, the early auditory potential further includes at least one of a compound action potential (CAP), a summating potential (SP), or an auditory nerve neurophonic (ANN).

In some implementations, the auditory prosthetic device is optionally a cochlear implant. In other implementations, the auditory prosthetic device is optionally an implantable or semi-implantable device.

A method for using early auditory potentials in an auditory prosthetic device is described herein. The method includes recording, using an electrode array, an early auditory potential, processing, using a digital signal processor (DSP), the early auditory potential to generate a stimulation signal, and transmitting the stimulation signal to the electrode array. The early auditory potential includes cochlear microphonic.

In some implementations, the method includes applying, using the electrode array, the stimulation signal within the subject's cochlea. Optionally, the electrode array is used to both record the early auditory potential and apply the stimulation signal.

In some implementations, the step of processing, using the DSP, the early auditory potential to generate the stimulation signal includes detecting and removing a stimulus artifact.

Optionally, the early auditory potential further includes at least one of a compound action potential (CAP), a summating potential (SP), or an auditory nerve neurophonic (ANN).

In some implementations, the electrode array is inserted into at least a portion of the subject's cochlea.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 illustrates how production of the cochlear microphonic (CM) is from current flow through channels in the stereocilia of hair cells as they open and close with movement in response to sound.

FIGS. 2A and 2B are diagrams illustrating a cochlear implant according to an implementation described herein. FIG. 2A illustrates the implantable prosthetic device. FIG. 2B is an enlarged view showing an electrode array including a plurality of electrodes (e.g., contacts #1-16) that can be used to record the CM and deliver electrical stimulation to the auditory nerve.

FIG. 3 is a block diagram illustrating an auditory prosthetic device according to an implementation described herein.

FIG. 4 is a block diagram illustrating an example computing device.

FIGS. 5A and 5B illustrate total response (mostly CM) for different subject groups. As shown in FIG. 5A, in CI subjects, children with ANSD (light grey) have among the largest responses, but some cases at all ages have similarly large responses, and almost all cases have some. As shown in FIG. 5B, distributions in other groups, including one subject with normal hearing. Notably, responses in these subjects with less hearing loss on average are largely overlapping with those of CI subjects.

FIG. 6 includes graphs illustrating electrocochleography to a speech signal. Top row: Time waveform and spectrum of HINT sentence—“He wore his yellow shirt.” Middle row: Responses to this sentence recorded from the round window of a normal hearing gerbil. Bottom row: Responses to the sentence recorded from the round window of a gerbil with a hearing loss mimicking that of a cochlear implant patient.

FIGS. 7A-7D illustrate an example method for removing stimulus artifact (from Koka and Litvak, 2017). FIG. 7A shows response to acoustic alone. FIG. 7B shows response to electrical alone, used as template for removal from combined stimulus. FIG. 7C shows response to combined stimuli. FIG. 7D shows recovered acoustic response after subtraction of the template compared acoustic alone.

FIGS. 8A-8F illustrate time and spectral domain representations of acoustic stimulus. FIGS. 8A-8C illustrate time and spectral domain representations of the acoustic stimulus /da/. FIGS. 8D-8F illustrate time and spectral domain representations of the acoustic stimulus /ba/. FIGS. 8A and 8D illustrate the acoustic stimuli in the time domain. FIGS. 8B and 8E are spectrograms, and FIGS. 8C and 8E illustrate the acoustic stimuli in the frequency domain. The arrows illustrate the spectrogram format structures (F₁-F₃) for both stimuli.

FIG. 9 is a table (Table 1) including demographic/surgical information of subjects who participated in a study of an implementation of the present disclosure. In the table, R=right, L=left, AAT=age at testing (years), RW=round window, ELS=endolymphatic sac decompression and shunt, CI=cochlear implant, VS=vestibular schwannoma, WRS=word recognition score, PTA=pure tone average. “*” indicates the participant who was diagnosed with auditory neuropathy spectrum disorder (ANSD).

FIG. 10 is an illustration of audiometric profiles for the study participants shown in FIG. 9. Squares represent for study participants who received cochlear implants, circles represent for study participants who were diagnosed with Meniere's disease and underwent endolymphatic sac decompression and shunt placement or labyrinthectomy, and diamonds represent for study participants who were having a vestibular schwannoma removed. NR refers to no response at the limits of the audiometer.

FIGS. 11A-11F illustrate exemplary ECochG_{diff} responses from study participants evoked by the /da/ and /ba/ stimuli. FIGS. 11A-11C illustrate ECochG_{diff} responses to /da/ stimulus for study participants A3, A7, and A9, respectively, shown in FIG. 9, and FIGS. 11D-11F illustrate ECochG_{diff} responses /ba/ for study participants A5, A1, and A4, respectively, shown in FIG. 9.

FIG. 12 is a table (Table 2) including evoked potential values of the difference waveform (ECochG_{diff}—subtraction of condensation and rarefaction raw waveforms) response values for stimuli /da/ and /ba/. “-” indicates that a trial for that subject was not carried out due to timing constraints during surgery. “*” indicates that the value was statistically significant (p<0.05).

FIG. 13 is an illustration of spectrograms of the normalized ECochG_{diff} evoked by an 80 dB nHL /da/ for the study participants shown in FIG. 9. The “Occluded Sound Tube” trial represents the average across all control trials where the sound tube was occluded with a hemostat and the stimulus presented at 80 dB nHL.

FIG. 14 is an illustration of spectrograms of the normalized ECochG_{diff} evoked by an 80 dB nHL /ba/ for some of the study participants shown in FIG. 9. The “Occluded Sound Tube” trial represents the average across all control trials where the sound tube was occluded with a hemostat and the stimulus presented at 80 dB nHL.

FIGS. 15A-15D illustrate results of Pearson correlations. FIGS. 15A-15B illustrate results of Pearson correlation between the preoperative pure tone average (PTA) and structural similarity index (SSIM) for /da/ (FIG. 15A) and /ba/ (FIG. 15B). The line in FIG. 15A indicates the line of best fit, r², for the significant correlation, and the line in FIG. 15B indicates a non-significant trend. The dot marked with an “X” in both plots represents the results of participant A4 who had auditory neuropathy spectrum disorder. FIGS. 15C-15D illustrate results of Pearson correlation between the SSIM and speech perception testing—word recognition score (WRS-%) for /da/ (FIG. 15C) and /ba/ (FIG. 15D). The lines in FIGS. 15C and 15D indicate the line of best fit, r² for significant correlations. As above, the dot marked with an “X” in both plots represents the results of participant A4 who had auditory neuropathy spectrum disorder.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for a cochlear implant, it will become evident to those skilled in the art that the implementations are not limited thereto, but are applicable for other auditory prosthetic devices including implantable devices.

The devices and methods described herein use early auditory potentials, especially the cochlear microphonic potential, produced by sensory hair cells in the cochlea and auditory neurons, as a type of fully implantable microphone to deliver acoustic input subsequently used for implantable hearing devices including, but not limited to, cochlear implant. For example, the ability to use early auditory potentials such as the cochlear microphonic as a microphone for an implantable hearing device is demonstrated by the examples below. Additionally, as described herein, the early auditory potentials can be recorded by an electrode array that is implanted in the subject's inner ear. In other words, the implanted electrode array is used for both electrical recording and electrical stimulation according to the methods and devices described herein.

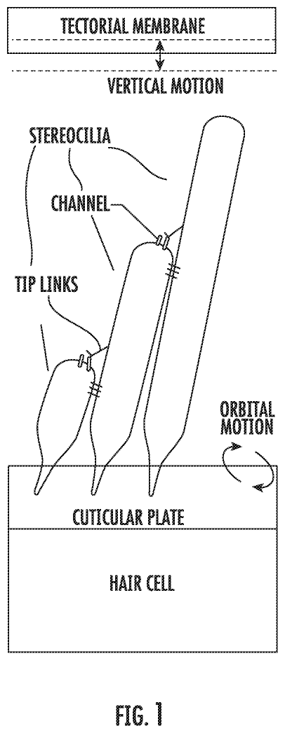

The cochlear microphonic (CM) is an electrical potential produced by sensory hair cells in the cochlea in response to sound [see FIG. 1]. Essentially, the stereocilia of hair cells bend back and forth in response to sound waves. Channels permeable to local cations open and close with the bending, producing an electrical current that preserves the structure of the input waveform. Thus, the CM is a useful, naturally occurring potential to collect sound information for cochlear implant stimulation. Further, some other early auditory potentials (e.g., summating potential [SP], auditory nerve neurophonic [ANN], and compound action potential [CAP]), arising mainly from auditory neurons (spiral ganglion cells), may also be utilized to trace back the original sound signal.

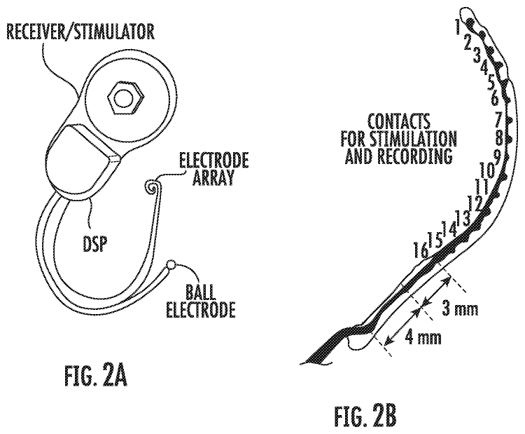

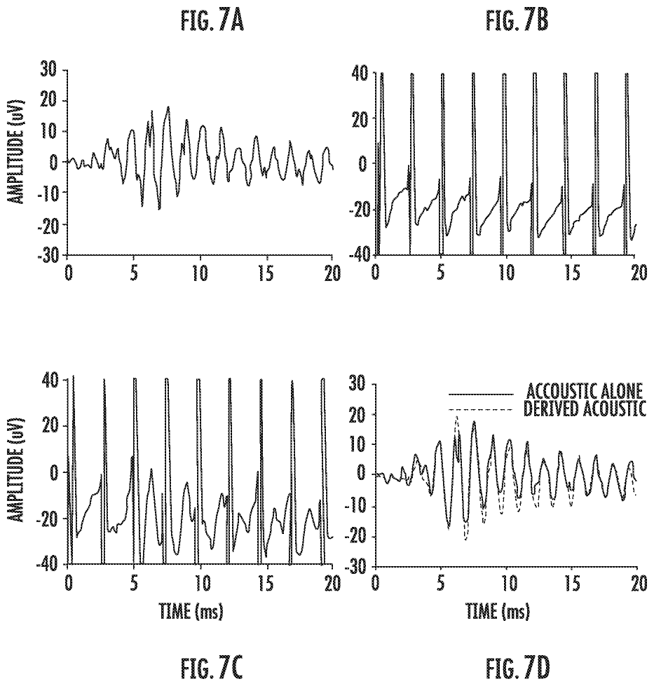

A cochlear implant includes an array of electrode contacts that is inserted into the cochlea [see FIGS. 2A and 2B]. As described herein, through signal processing techniques the output of a microphone is converted to electrical stimulation of each contact in a frequency specific manner simulating the natural place-specific frequency arrangement of the cochlea (i.e, low frequencies are delivered to more apical contacts and high frequencies to basal contacts, also termed tonotopy). The CM can be recorded by the same array that is used to produce electrical stimulation and can thereby provide the microphone input that can be used to drive electrical stimulation, all within the digital signal processor (DSP) of the implanted array [FIG. 7].

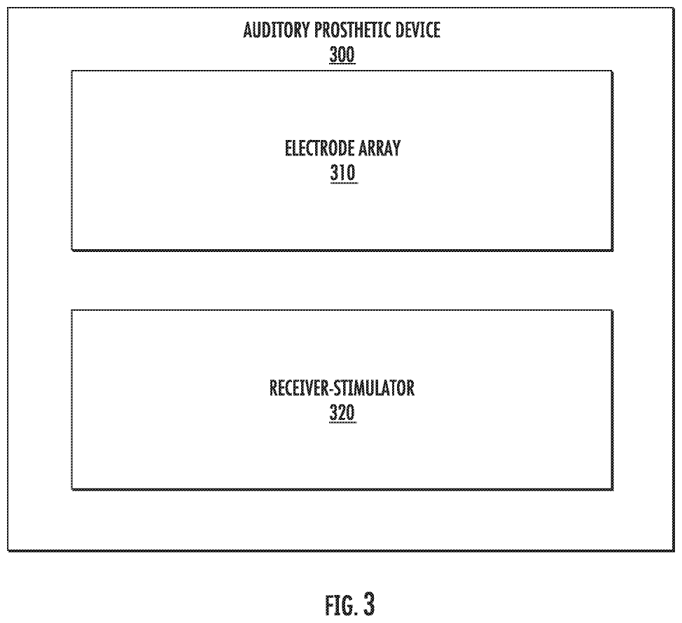

Referring now to FIG. 3, an example auditory prosthetic device 300 is described. The auditory prosthetic device 300 can include an electrode array 310 that is configured for implantation into a subject's inner ear, and a receiver-stimulator 320 operably coupled to the electrode array 310. In some implementations, the receiver-stimulator 320 is optionally implanted in the subject's body. For example, the electrode array 310 can be inserted into at least a portion of the subject's cochlea. This disclosure contemplates that the electrode array 310 can record early auditory potentials either inside or outside of the subject's cochlea. In some implementations, the electrode array 310 is partially inserted into the subject's cochlea. In other implementations, the electrode array 310 is completely inserted into the subject's cochlea. The electrode array 310 and the receiver-stimulator 320 can be coupled by a communication link. This disclosure contemplates the communication link is any suitable communication link. For example, a communication link may be implemented by any medium that facilitates signal exchange between the electrode array 310 and receiver-stimulator 320. In some implementations, the auditory prosthetic device 300 is a cochlear implant. An example cochlear implant is shown in FIGS. 2A and 2B. Although the implementations are described below with regard to cochlear implants, it should be understood that the auditory prosthetic device 300 can be an implantable device such as a fully-implantable prosthetic device or a semi-implantable prosthetic device.

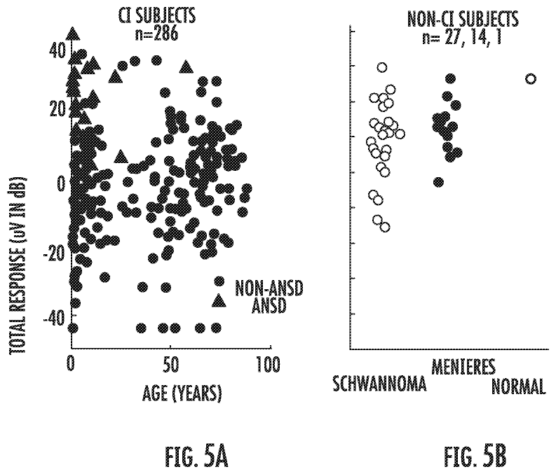

As described herein, the electrode array 310 can be configured for electrical recording and stimulation. This is different than conventional cochlear implants where a microphone, which is located externally with respect to the subject's body, records sound, which is then processed by a sound/speech processing unit worn by the subject (e.g., clipped to clothing or hooked behind the ear) and also located externally with respect to the subject's body. In the conventional cochlear implant, the processed sound signal is then transmitted to a receiver-stimulator (e.g., receiver-stimulator 320), which is implanted inside the subject's body. The microphone and/or sound/speech processing unit can be coupled to the implanted receiver-stimulator with a magnet. The receiver-stimulator then converts the processed sound signal into a stimulation signal, which is transmitted to an electrode array (e.g., electrode array 310) arranged within the subject's cochlea. Thus, the electrode array in a conventional cochlear implant is driven by sound recorded by an external microphone. Unlike a conventional cochlear implant, the auditory prosthetic device 300 described herein uses an early auditory signal (e.g., a cochlear potential such as the CM), which is recorded by the electrode array 310 arranged within the subject's cochlea, to drive stimulation. In other words, the electrode array 310 (i.e., the same electrode array, which is implanted in the subject's cochlea) is used for both recording electrical activity within the subject's cochlea and also applying the electrical stimulation within the subject's cochlea. As described below and shown by FIGS. 5A and 5B, the CM is often present, and can be large, even in subject's with hearing impairments. Accordingly, the CM can be recorded and used to drive stimulation. This allows the provision of a fully-implantable microphone without external components.

The electrode array 310 can include a plurality of electrodes (sometimes referred to herein as “contacts”) (e.g., as shown in FIGS. 2A and 2B). The electrodes of the electrode array 310 can be arranged to correspond to different tonotopic locations within the subject's cochlea. The number and/or arrangement of the contacts shown in FIG. 2B are provided only as an example. This disclosure contemplates that the number and/or arrangement of contacts may be different than those shown in FIG. 2B. It should be understood that the cochlea allows perception of sounds in a wide frequency range (e.g., ^{˜}20 Hz to ^{˜}20 kHz). Different portions of the cochlea move in response to different frequencies, for example, lower frequencies cause movement near the apex while higher frequencies cause movement near the base. Each of the electrodes of the electrode array 310 therefore records a different spectral component due to its respective tonotopic location. This disclosure contemplates that a respective potential can be recorded at each of the one or more electrodes. As described herein, the electrode array 310 can record the early auditory potential within the subject's cochlea, e.g., the electrical potential that arises naturally in the subject's cochlea through activity of sensory cells and auditory neurons. The early auditory potential can include CM, which is produced by sensory hair cells in the cochlea. It should be understood that CM can be the dominant component of the early auditory potential. The early auditory potential, however, can include other components, e.g., other potentials arising naturally in the subject's cochlea. These other potentials can include, but are not limited to, a compound action potential (CAP), a summating potential (SP), and/or an auditory nerve neurophonic (ANN). In some implementations, the early auditory potential can be recorded at one or more of the electrodes of the electrode array 310. In other implementations, the early auditory potential can be recorded at each of the electrodes of the electrode array 310 (i.e., all of the electrodes of the electrode array 310). As described herein, the early auditory potential can be processed to generate a stimulation signal. Optionally, in some implementations, the early auditory potential can be recorded (e.g., sampled) a plurality of times and then combined, for example averaged. This disclosure contemplates obtaining an averaged early auditory potential at each of the one or more electrodes of the electrode array 320. The averaged early auditory potential signal can be used as the microphone.

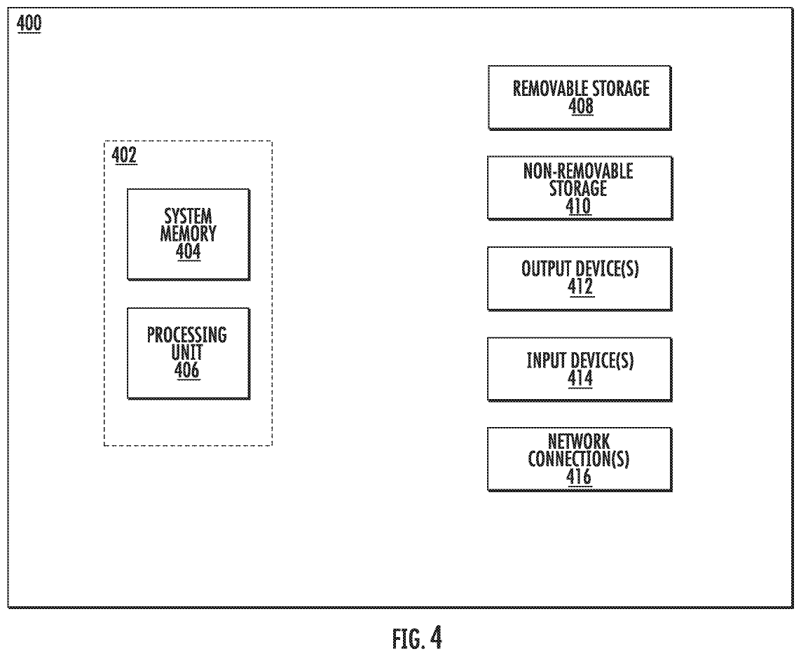

The receiver-stimulator can include the device's circuitry, including a digital signal processor (DSP). A DSP is a specialized microprocessor (e.g., including at least a processor and memory as described with regard to FIG. 4) for signal processing. Signal processing can include, but is not limited to, analog-to-digital conversion (ADC), filtering, compression, etc. of analog signals such as the early auditory potential (e.g., including CM) recorded by the electrode array 310. DSPs are known in the art and are therefore not described in further detail herein. The DSP of the receiver-stimulator 320 can be configured to receive the early auditory potential recorded within the subject's cochlea (e.g., the cochlear microphonic), process the early auditory potential to generate a stimulation signal, and transmit the stimulation signal to the electrode array 310. This disclosure contemplates that the early auditory potential recorded at each respective electrode can be converted to a respective stimulation signal for each of the electrodes of the electrode array 310 in a frequency-specific manner. A respective electrode position along the electrode array 310 determine pitch (e.g., frequency), and a current level determines the loudness. This simulates the natural location-specific frequency arrangement of the subject's cochlea (e.g., lower frequencies delivered to apical electrodes/higher frequencies delivered to basal electrodes). As described herein, the stimulation signal(s) can be applied within the subject's cochlea using the electrode array 310.

In some implementations, the step of processing the early auditory potential to generate the stimulation signal can include detecting and removing a stimulus artifact. This disclosure contemplates that stimulus artifact can be detected and removed using one or several of various techniques. Artifact detection and removal are known in the art and this disclosure contemplates using such known techniques with the devices and methods described herein. For example, one such technique is a template matching technique (Koka and Litvak, 2018) described below and also shown in FIGS. 7A-7D. Others techniques include, but are not limited to, low pass filtering (see Litvak, L., B. Delgutte and D. Eddington (2003). “Improved neural representation of vowels in electric stimulation using desynchronizing pulse trains.” J Acoust Soc Am 114(4 Pt 1): 2099-2111), blanking of the recording amplifier at the time of the artifact to avoid amplifier saturation with interpolation of the response during the blanked interval (see Hofmann, M. and J. Wouters (2010). “Electrically evoked auditory steady state responses in cochlear implant users.” J Assoc Res Otolaryngol 11(2): 267-282), or using an amplifier with sufficient bandwidth and sensitivity that the fast artifact is not smeared in time and does not saturate the amplifier, while the much smaller CM remains detectable (U.S. Pat. No. 6,195,585).

Optionally, early auditory potentials other than the CM can be used to drive the stimulation. Such early auditory potentials can include, but are not limited to, the compound action potential (CAP) from the auditory nerve which signals stimulus onsets or transient sounds, the auditory nerve neurophonic (ANN), also from the auditory nerve which follows the waveforms of low frequency sounds, and the summating potential (SP), which is proportional to the signal envelope rather than the fine structure. This disclosure contemplates that the CAP, ANN and/or the SP can be recorded using the electrode array 310. Although CAP, ANN, and SP are provided as examples, the early auditory potentials can include any potential arising naturally in the subject's cochlea through activity of sensory cells and auditory neurons as described herein. While CM most faithfully follows the sound waveform, this disclosure contemplates recording and using other early auditory potentials to improve the stimulation pattern to more faithfully represent the information provided to the auditory nerve by cochlear processing, in addition to the degree of faithful (linear) processing encoded in the sound waveform.

An example method for using early auditory potentials in an auditory prosthetic device (e.g., auditory prosthetic device 300 of FIG. 3) is also described herein. The method can include recording, using an electrode array (e.g., electrode array 310 of FIG. 3), an early auditory potential within a subject's cochlea; processing, using a DSP (e.g., DSP of receiver-stimulator 320 of FIG. 3), the early auditory potential to generate a stimulation signal; and transmitting the stimulation signal to the electrode array. Additionally, the method can include applying, using the electrode array, the stimulation signal within the subject's cochlea. As described herein, the electrode array (i.e., the same electrode array) can be used to both record the early auditory potential and apply the stimulation signal.

It should be appreciated that the logical operations described herein with respect to the various figures may be implemented (1) as a sequence of computer implemented acts or program modules (i.e., software) running on a computing device (e.g., the computing device described in FIG. 4), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 4, an example computing device 400 upon which the methods described herein may be implemented is illustrated. It should be understood that the example computing device 400 is only one example of a suitable computing environment upon which the methods described herein may be implemented. Optionally, the computing device 400 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 400 typically includes at least one processing unit 406 and system memory 404. Depending on the exact configuration and type of computing device, system memory 404 may be volatile (such as random access memory (RAM)), non-volatile (such as read-only memory (ROM), flash memory, etc.), or some combination of the two. This most basic configuration is illustrated in FIG. 4 by dashed line 402. The processing unit 406 may be a standard programmable processor that performs arithmetic and logic operations necessary for operation of the computing device 400. The computing device 400 may also include a bus or other communication mechanism for communicating information among various components of the computing device 400.

Computing device 400 may have additional features/functionality. For example, computing device 400 may include additional storage such as removable storage 408 and non-removable storage 410 including, but not limited to, magnetic or optical disks or tapes. Computing device 400 may also contain network connection(s) 416 that allow the device to communicate with other devices. Computing device 400 may also have input device(s) 414 such as a keyboard, mouse, touch screen, etc. Output device(s) 412 such as a display, speakers, printer, etc. may also be included. The additional devices may be connected to the bus in order to facilitate communication of data among the components of the computing device 400. All these devices are well known in the art and need not be discussed at length here.

The processing unit 406 may be configured to execute program code encoded in tangible, computer-readable media. Tangible, computer-readable media refers to any media that is capable of providing data that causes the computing device 400 (i.e., a machine) to operate in a particular fashion. Various computer-readable media may be utilized to provide instructions to the processing unit 406 for execution. Example tangible, computer-readable media may include, but is not limited to, volatile media, non-volatile media, removable media and non-removable media implemented in any method or technology for storage of information such as computer readable instructions, data structures, program modules or other data. System memory 404, removable storage 408, and non-removable storage 410 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 406 may execute program code stored in the system memory 404. For example, the bus may carry data to the system memory 404, from which the processing unit 406 receives and executes instructions. The data received by the system memory 404 may optionally be stored on the removable storage 408 or the non-removable storage 410 before or after execution by the processing unit 406.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

Scientific Basis

The CM in cochlear implant subjects (candidates): At first glance it might appear that the CM would only provide information already available to the subject in the form of hearing, so what would be the usefulness of converting this information to electrical stimulation? And, since they have hearing loss sufficient to need a cochlear implant, why would there even be a CM to be recorded? The answers to these questions lie in recent findings that the CM in cochlear implant subjects can be large even in cases of a severe hearing loss. One etiology of hearing loss where this result is clear is in children with auditory neuropathy spectrum disorder (ANSD), defined as loss of activity in the auditory nerve that is independent of cochlear function. In these subjects, the CM is exceptionally large, even though hearing is poor [Riggs, W. J., et al., Intraoperative Electrocochleographic Characteristics of Auditory Neuropathy Spectrum Disorder in Cochlear Implant Subjects. Front Neurosci, 2017. 11: p. 416]. However, the CM is also large in children and adults not diagnosed as auditory neuropathy, where mechanisms such as cochlear synaptopathy, or destruction of the synapses between hair cells and the auditory nerve by overstimulation, can lead to greater loss of neural activity relative to that of hair cells [Kujawa, S. G. and M. C. Liberman, Adding insult to injury: cochlear nerve degeneration after “temporary” noise-induced hearing loss. J Neurosci, 2009. 29(45): p. 14077-85; Liberman, M. C. and S. G. Kujawa, Cochlear synaptopathy in acquired sensorineural hearing loss: Manifestations and mechanisms. Hear Res, 2017. 349: p. 138-147]. Thus, the long-held view that hearing loss is primarily caused by loss of sensory hair cells appears not entirely correct. Instead, hair cell potentials often remain quite robust despite the loss of hearing.

To support this new view, the distribution of cochlear responses is shown for various groups of subjects, all recorded intraoperatively with an electrode at the round window of the cochlea where the signal to noise ratio is highly favorable. The metric is the total response (TR), which is the sum of all response magnitudes recorded to tones of different frequencies [Fontenot, T. E., et al., Residual Cochlear Function in Adults and Children Receiving Cochlear Implants: Correlations With Speech Perception Outcomes. Ear Hear, 2018]. Although the TR includes both CM and neural responses, the CM typically dominates [Fontenot, T. E., C. K. Giardina, and D. C. Fitzpatrick, A Model-Based Approach for Separating the Cochlear Microphonic from the Auditory Nerve Neurophonic in the Ongoing Response Using Electrocochleography. Front Neurosci, 2017. 11: p. 592]. When recording from outside the cochlea at the round window (RW), the largest group in FIG. 3A are cochlear implant subjects. Within this group those with the largest responses include children with auditory neuropathy spectrum disorder (ANSD), but other ages including the elderly also feature many cases with large responses. Across all age groups, reasonable response magnitudes are obtained from nearly all subjects (280/286, six subjects plotted at 94 dB had no responses). In the devices and methods described herein, the recordings of the CM can be intracochlear, where the magnitudes of the responses increase by a large proportion (typically 2-10 times). The next groups (FIG. 5B) are subjects undergoing labyrinthectomy for Meniere's disease or removal of a vestibular schwannoma. Both of these groups can expect a lesser degree of hearing loss on average than CI subjects, and in some cases to have reasonably good hearing. There is also one subject included with normal hearing who was undergoing surgery for removal of a tumor near the jugular foramen. Most importantly, looking across FIGS. 5A and 5B the CMs of CI subjects span a wide range that overlaps greatly with those where the hearing loss is less, including many with responses difficult to distinguish from normal. Thus, there is strong evidence that the CM in cochlear implant subjects is available to be recorded and used to drive stimulation.

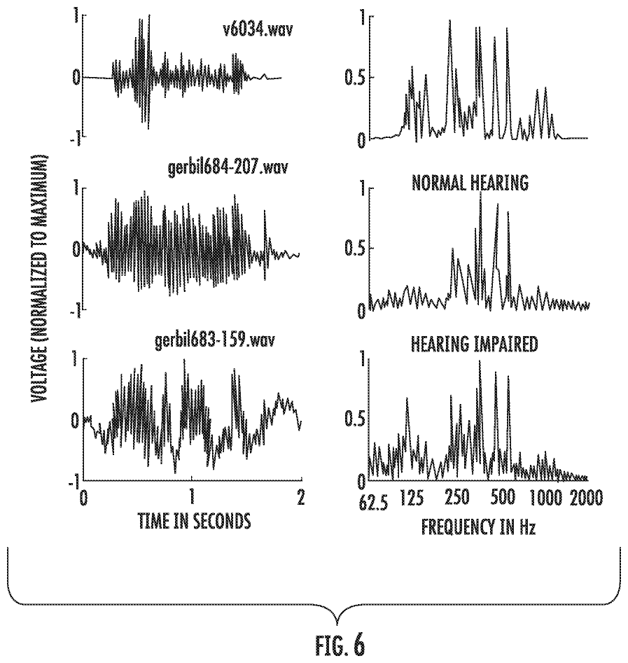

Pre-clinical evidence that the CM accurately represent speech: That speech can be obtained by the CM is shown through the familiar experiment where someone speaks into an animal's ear, the CM is recorded and fed directly to a speaker outside the sound-proof booth, and listeners outside can readily understand what was said on the inside [Wever, E. G. and C. Bray, Action currents in the auditory nerve in response to acoustic stimulation. Proc. Nat. Acad. Sci., U.S. A., 1930. 16: p. 344-350]. In animal models, the CM can be recorded in normal hearing animals and in animals with various degrees of hearing loss. A speech pattern of a HINT sentence commonly used in speech tests is shown in both time and frequency domains in the top row of FIG. 6. A round-window recording of cochlear potentials to a single presentation of this stimulus in a normal hearing animal is shown by the graphs in the middle panel. The CM is the largest component of the response. The waveform is distorted but the spectrum is relatively preserved, and when played back the sentence is easily interpretable by a human listener (included). The bottom panel shows a recording from an animal treated with ototoxins that remove a large proportion of outer hair cells primarily from basal and middle regions of the cochlea. This is a pattern common to subjects with hearing loss including those needing cochlear implants. Even with this high degree of hearing loss the spectrum remains similar to the stimulus although reduced in size, and the sentence is readily understood (included). This bottom panel was recorded from 10 repetitions instead of 1 to remove the heartbeat, which was detectable due to the smaller size of the response. The sentence could still be understood even to 1 repetition (included).

Recording and analyzing the CM to provide information useful to CI recipients: In human subjects, the CM can be recorded by all of the contacts on the array. The contacts on the array record different spectral components because they are at different tonotopic locations. This information about tonotopic location of each contacts is not available from other technologies and will greatly assist in mapping sound frequencies to each contact.

One issue is that with combined recording and stimulation the electrical artifacts (e.g., stimulus artifacts) imposed by large current pulses could compromise the recording of the CM. However, with modern signal processing techniques and an optimized stimulus/recording platform this difficulty can be overcome. The platform can be of high enough bandwidth to handle the large amplitude stimulation pulses without saturation to avoid time-smearing. The artifact is short, on the order of 100 microseconds, and its timing is well-defined since it is generated within the device using the same clock as the recording system. It comes at a low enough rate (about 1 kHz) so that approximately 90% of the recording time is signal and not artifact. The shape of the recorded artifact can therefore be stored and removed as a template, for example, as reported in Koka and Litvak, 2018. In this case the template of electrical stimulation alone was recorded immediately before the combined electrical and acoustic stimulation, and when subtracted from the combined stimulus the response to acoustic stimulation was recovered. In the case where the CM was used as the microphone a scalable template of electrical stimulation can be stored and subtracted based on the timing of the electrical stimulation to achieve a running record of acoustic response within the electrical stimulation. Alternatively, the period containing the artifact can be removed and filled-in by interpolation of the remaining response.

In addition, not all recording and stimulation will be in challenging environments such as a conversation. There are many instances where simple alerts as to the presence of environmental sounds, such as when sleeping with the external contact removed but needing to hear a crying baby, a phone call, or fire alarm. In this case simply maintaining a representation of background levels requires a much lower level of timing precision.

Applications

The devices and methods described herein can be used to record sound input and provide stimulation patterns that support speech perception. Additionally, the devices and methods described herein can be used for stimulation in listening situations where the user is not wearing their external components, such as when sleeping or when taking a shower. Other potential applications for the devices and methods described herein include, but are not limited to:

Use of the cochlear implant electrode as the microphone. In this application, the CI electrode can be placed in a standard fashion, preferably using a hearing preservation technique. The potential can then be recorded through one or multiple electrode contacts from the implant and used as the sound source for the subsequent signal processing (thus the microphone). Since the stimulation will be intracochlear as well, the sensing and stimulation portions will need to be sequential.

Use of a separate intra- or extracochlear (preferably RW) electrode to act as the sound sensor (microphone). These potential applications certainly also include a CI but could be expanded to include fully or semi-implantable hearing devices such as active middle ear implants. In this application, the sound sensing can pick up the signal and send it to either an implanted or externally worn processor for subsequent signal processing before the stimulus is delivered.

Use of the cochlear implant electrode as a second microphone in conjunction with the input through the external speech processor.

Utilizing Electrocochleography as a Microphone for Fully Implantable Cochlear Implants.

Current cochlear implants (Cis) are semi-implantable devices with an externally worn sound processor that hosts the microphone and sound processor. A fully implantable device, however, would be desirable as it would be of great benefit to recipients. While some prototypes have been designed and used in a few select cases, one main stumbling block is the sound input. Specifically, subdermal implantable microphone technology has been poised with physiologic issues such as sound distortion and signal attenuation under the skin. An alternative is disclosed herein that utilizes a physiologic response composed of an electrical field generated by the sensory cells of the inner ear to serve as a sound source microphone for fully implantable hearing technology such as Cis. Electrophysiological results obtained from 14 participants (adult and pediatric) document the feasibility of capturing speech properties within the electrocochleography (ECochG) response. Degradation of formant properties of the stimuli /da/ and /ba/ are evaluated across various degrees of hearing loss. The results suggest that using the ECochG response as a microphone is feasible to capture vital properties of speech.

To date, it is estimated that as many as 466 million individuals worldwide have hearing loss as defined as an average hearing level of 235 dB HL by pure-tone audiometry [1]. Treatment options for hearing loss typically depend on the severity of the hearing loss. Cochlear implants (CI) have long been a treatment option for individuals with severe-to-profound hearing loss; however, with advancements in technology, candidacy criteria have expanded to include individuals with greater amounts of residual hearing. With this trend, the focus has shifted toward developing techniques and technology to allow for the preservation of residual hearing, as this has been shown to be important in obtaining optimal outcomes through the use of electric-acoustic stimulation. That is, in patients who receive CIs but maintain some useable residual hearing, the implanted ear can be stimulated using the ipsilateral combination of electric (CI) and acoustic (hearing aid) [2] [3].

In attempts to achieve preservation of residual hearing, implementation of electrocochleography (ECochG) at the time of CI surgery has recently been described. ECochG is a tool that allows electrophysiological assessment of the peripheral auditory system (i.e., the cochlea and auditory nerve) by using acoustic stimulation. Specifically, ECochG has been used as a monitoring tool during CI surgery in an effort to provide real-time feedback of inner ear physiology that allows for modifying surgical technique in an attempt to avoid trauma caused by the electrode insertion, hence preserving residual hearing [4, 5, 6]. Technology has recently been introduced that allows the ECochG signal to be recorded through the CI electrode array (CI-ECochG). This technique has great advantages: recording ECochG through an electrode contact of the CI provides favorable signal-to-noise ratios allowing for the acquisition of physiologic data in real-time even in ears with substantial degrees of hearing loss [5, 6]. In addition to its surgical utility, this technique can accurately predict post-operative behavioral audiometric thresholds of residual hearing [7].

Current standard CI systems contain two main components: (1) an internal electrode array and receiver stimulator which are implanted at the time of surgery and (2) an external speech processor with a coil headpiece which connects to the internal portion via electromagnetic induction [8]. The battery source is external and connected to the speech processor that is typically worn over the ear. The speech processor contains multiple microphone configurations that detect acoustic information of the external environment. While batteries have been developed for fully implantable CIs (meaning no external components are required), a main barrier to fully implantable devices is how to capture sounds when there is no external microphone component. To address this issue, various implantable microphone concepts have been explored in the past, such as subcutaneous devices [9] and options which obtain mechanical sound energy from the middle ear ossicular chain [10]. However, each of these options has drawbacks, including interference from body noise, substantial attenuation of sound signals, high power consumption, and difficult calibration. However, fully implantable CIs are highly desirable and of great clinical relevance to recipients, permitting around-the-clock CI use, enhanced safety due to constant access to sound input, improved cosmetics, and the ability to participate in water sport activities without special water-proof equipment.

The systems and methods described herein include application of CI-ECochG technology, relying on the cochlear microphonic (CM) response of the inner ear (e.g., an early auditory potential), configured to serve as an implanted, internal microphone for CI devices. The ECochG signal is a gross evoked potential that is dominated by cochlear hair cell activity represented by the CM and summating potentials [11, 12] as well as contributions from neural sources [13, 14]. The CM is thought to predominately reflect evoked activity of hair cells (outer) due to basilar membrane motion, with its morphology alternating in polarity and following the phase of the incoming signal [15]. First described by Weaver and Bray [16], it was termed the ‘microphone’ potential as the response typically mimics the acoustic waveform generated from an external source that is transferred from the external ear canal and middle ear. Owing to this property, the CM response could serve as an internal microphone for a hearing device such as a CI. Thus, this property of the CM obtained in the ECochG response can be used to back-trace the acoustic properties of the original sound signal. That is, recording the CM response from an intracochlear electrode and subsequently processing the response (e.g., as an external CI speech processor would do in a conventional CI) and delivering it to the internal receiver stimulator as a representation of the acoustic signal that was delivered to the ear, supports the development of an implantable microphone as a vital component of a fully implantable CI. However, while CI-ECochG platforms are clinically available, the use of this technology as a microphone is not available in conventional CI platforms. Thus, the current study employed an extracochlear round window (RW) approach to demonstrate proof-of-concept for this potential application in future CI technology.

In order to utilize ECochG CM responses as an internal microphone, the resulting signal's quality can be a desirable property to optimize. Specifically, in implementations utilizing the CM response as a microphone that is ultimately used to drive stimulation of the CI, speech information (acoustic features/properties) should be preserved within the ECochG response. Since the CM response is dominated by hair cell activity [17], sensorineural hearing loss (SNHL) would likely cause degradation of how well the incoming signal is represented by the CM. Therefore, one objective of this study was to assess the ability of the CM to accurately represent a speech signal in ears with SNHL.

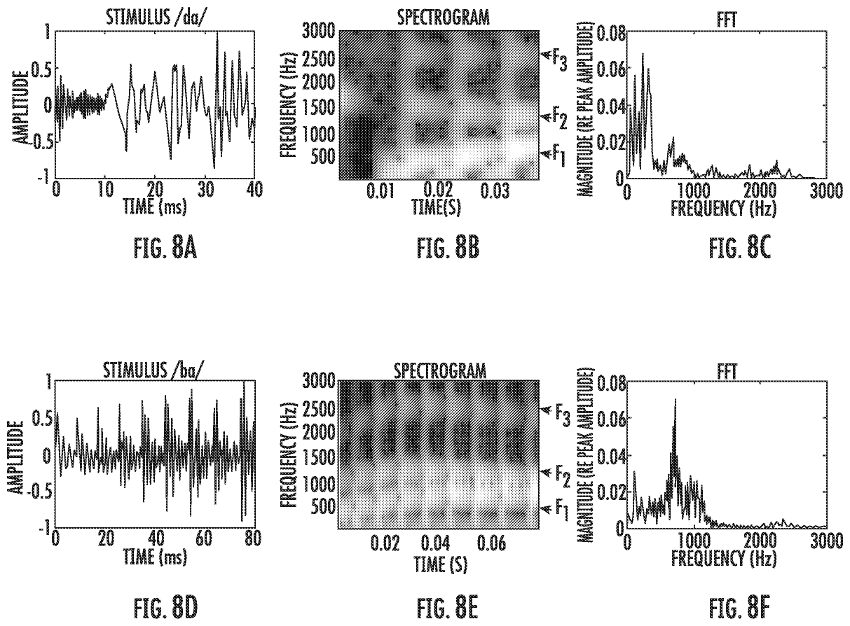

An important acoustic property of a speech signal is the formant structure. Formants are concentrated regions of energy that represent the acoustic resonances of the vocal tract [18]. That is, the glottal folds generate a fundamental frequency (pitch—F₀) and the resonances of the vocal tract following glottal vibration create a multi-formant structure numbered in an upward fashion (F1, F2, F3 . . . ) as frequency bands increase. F₀ are important for identification of pitch of the voice while the acoustic behavior of the formants following F₀ are critical for identification/differentiation of the speech sound [19, 20]. As a main objective was to determine the feasibility of utilizing the ECochG signal as a microphone, how representation (frequency and intensity) of the formant structure (F₁-F₃) behaved in various degrees of SNHL using vowel-dominated phonemes (FIGS. 8A-8F) when recorded from the RW was evaluated. With SNHL, it is expected that both amplitudes (valley-peak) and frequency resolution are reduced due to fewer sensory hair cells and broadened auditory filters [21]. Perceptually, vowel identification has been reported to be fairly robust even in ears with significant degrees of hearing loss [22, 23, 24, 25]. Thus, it is predicted that a substantial portion of the formant structure would be encoded adequately at the level of the cochlear hair cells despite significant degrees of hearing loss.

The present report evaluated the ECochG response's capability, when recorded from the RW of the cochlea, to represent formant structure of an acoustic speech signal in humans undergoing a variety of otologic surgeries with diverse preoperative hearing conditions. That is, both CI participants (severe hearing loss) and non-CI participants (mild-to-severe) were included to establish how well the speech signal can be represented by the EcochG and evaluate its degradation with increasing severity of hearing loss.

### Experimental Results

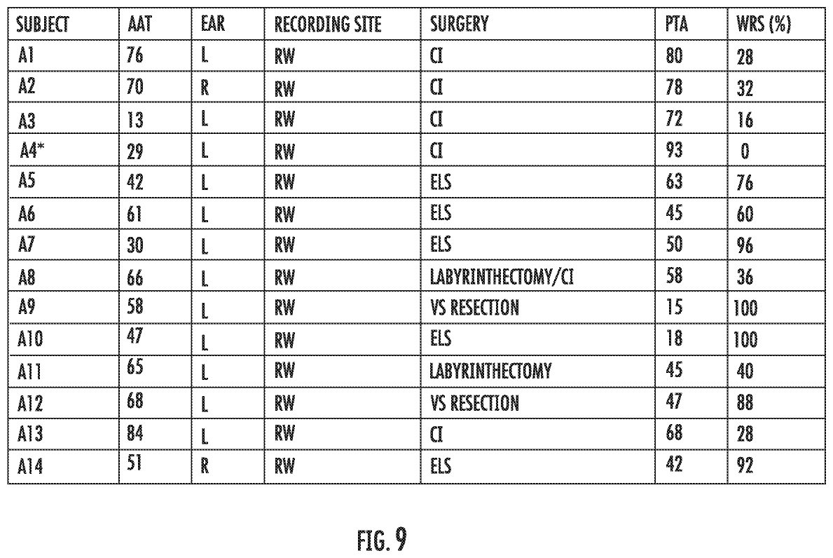

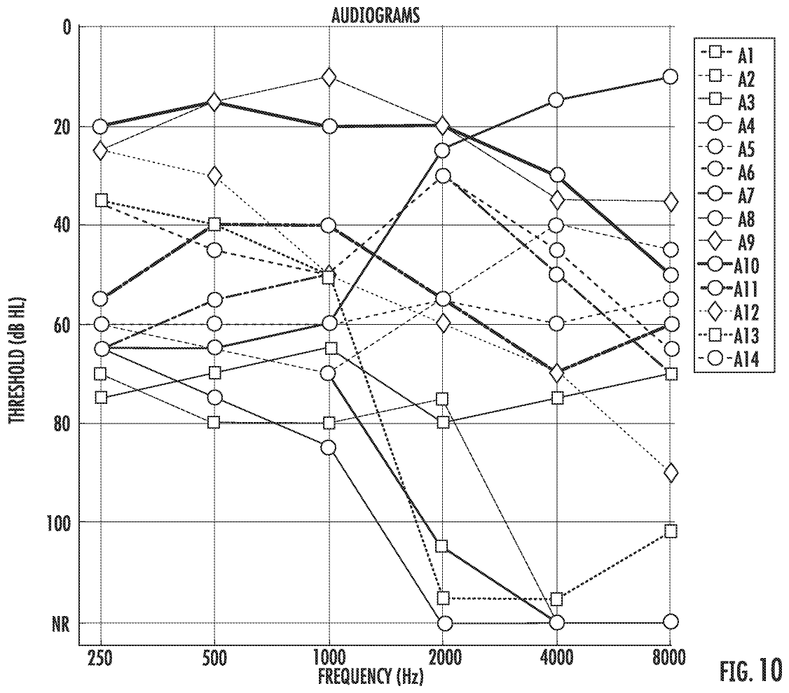

Hearing Profiles. Demographic and surgical information of study participants (n=14) can be found in FIG. 9 (Table 1). Results of audiometric testing near the time of surgery can be seen in FIG. 10. The study group exhibited widespread audiometric thresholds ranging from mild to profound SNHL. Pure tone average (PTA—0.5, 1, 2 kHz) ranged from 15-93.33 dB HL (mean: 56.21 dB, SD: 24.8 dB) with word recognition scores (WRS) ranging from 0-100% (mean: 45.45%; SD: 37.41%). Note, participant A4 was diagnosed with auditory neuropathy spectrum disorder (ANSD), previously shown to have robust cochlear function exhibited by large CM responses but neural dyssynchrony and 0% WRS.

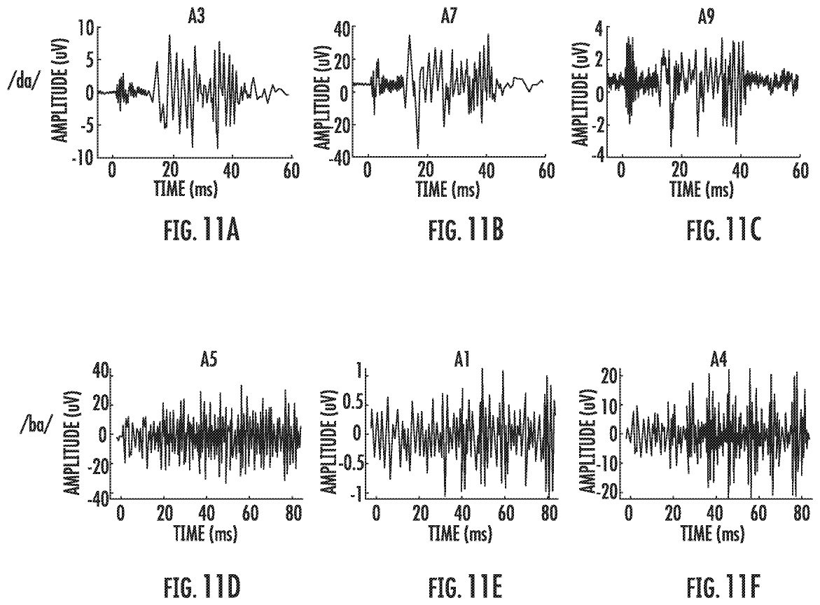

Electrophysiology representation of the stimulus: Time domain. To emphasize components of the ECochG response that change with stimulus phase, such as the CM dominated portion, a difference waveform (ECochG_{diff}) was created by subtracting the ECochG response evoked by the rarefaction phase from the ECochG response evoked by the condensation phase. Base-to-peak amplitudes (μV) of the non-normalized ECochG_{diff} response (time domain), measured as the region of the ECochG_{diff} response after stimulus onset that produced the maximal amplitude deflection, were calculated and for those evoked by /da/presented at 108 dB peak equivalent sound pressure level (peSPL) ranged from 2.46-46.06 μV (n: 14, mean: 13.73 μV, SD: 13.43 μV). Amplitudes for the /ba/ responses presented at 89 dB peSPL ranged from 1.10-29.80 μV (n: 11, mean: 9.60, SD: 9.53). The difference in peaks was expected as the overall peak sound pressure level (peSPL) value for the /da/ was 19 dB louder than the /ba/. Examples of raw ECochG_{diff} responses for both stimuli can be seen in FIGS. 11A-11F. In comparison to the time domain waveforms of the stimuli (FIGS. 8A and 8D), visually, the overall fidelity (time domain representation of the stimulus) appears to be maintained in the ECochG_{diff} response. Of note, largest amplitudes were observed in participants with the diagnosis of Meniere's disease (MD) while smallest amplitudes were typically exhibited by those receiving a CI (without MD diagnosis).

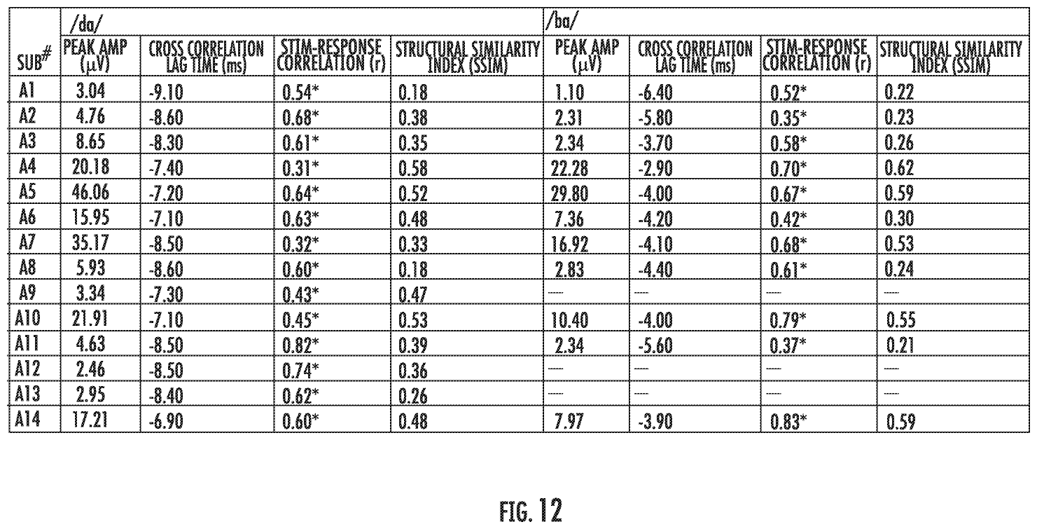

Each ECochG_{diff} response was then normalized to its peak amplitude (maximal voltage of the time domain response) for each individual participant. Following normalization, as ECochG is an evoked response, it was necessary to align (i.e. adjust in latency or lag time) the evoked ECochG_{diff} response with that of the stimulus. This was achieved with a cross-correlation approach that yielded a latency (lag time) value (ms) where the two waveforms (stimulus and ECochG response) were most highly correlated. ECochG_{diff} latency times ranged from −9.10 to −6.90 ms (mean: −7.96, SD: 0.75) for the /da/ and −6.40 to −2.90 ms (mean: −4.45, SD: 1.04) for the /ba/. Latency values were based on a single ECochG_{diff} trial for each participant and variation in lag time was expected due to the different severities of SNHL across the study group. After adjusting for lag time, Pearson product-moment correlation was run between the stimulus and each ECochG_{diff} response. All correlations were found to be statistically significant (p<0.05) and their coefficients are shown in FIG. 12 (Table 2). Coefficients ranged from 0.31-0.82 (mean: 0.57, SD: 0.15) and 0.35-0.83 (mean: 0.59, SD: 0.16) for the /da/ and /ba/respectively as shown in FIG. 12. Overall, this suggested a moderate to strong correlation (i.e. waveform similarity) after alignment between each ECochG_{diff} response and the stimulus based on its time domain representation for both stimuli.

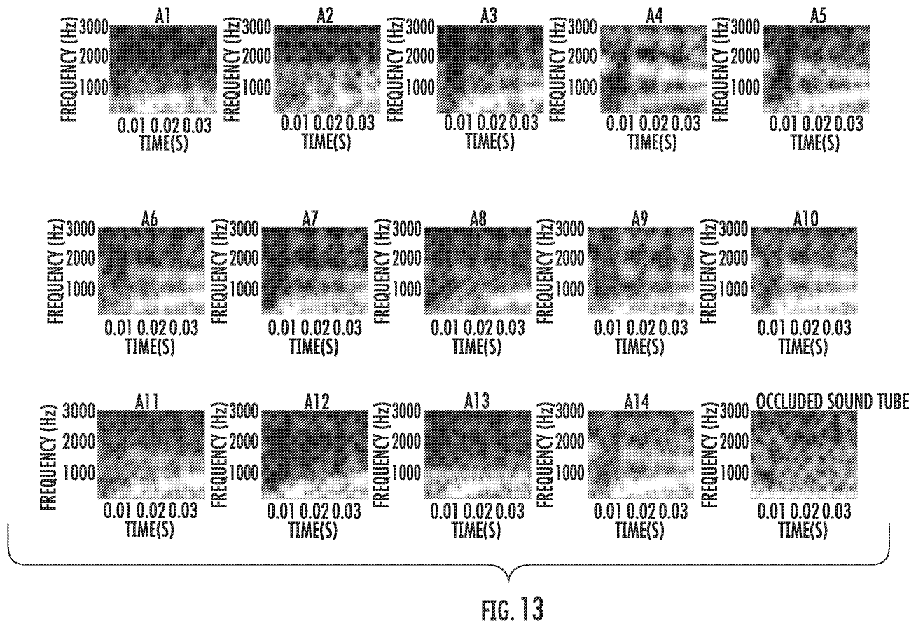

Electrophysiology representation of the stimulus: Spectrogram. To evaluate representation of the stimulus formant frequencies over time that were present in the ECochG_{diff} response, each response was windowed into segments composed of 240 points and fast Fourier transforms (FFTs) were then used to create spectrograms of the normalized lag time aligned ECochG_{diff} responses. Spectral amplitude at the center frequency of each formant was calculated at three regions along the formant (beginning, middle, end) to determine significance above the noise floor (see Methods below). If all points along each formant band were significant then this was considered full formant representation. If only one or two regions were significant per formant, then partial representation was considered. The spectrograms for each subject are shown in FIG. 13 and results of the FFT analyses indicated that the formant structure of the /da/ evoked ECochG_{diff} varied in its representation across the responses of the study group. Overall, 13 participants had full F₁ representation present in the ECochG_{diff} response and one (A8) had a partial F₁. Eight participants had both full F₁ and F₂ representation of which three (A4, A7, A9) also had full representation of all three formants while three had partial (A5, A6, A10). One participant had full F₁ with partial F₂ present (A11) and four participants (A1, A2, A12, A13) had only an F₁ structure present. The averaged occluded sound tube response trial (sound tube clamped by a hemostat) can be seen in the last panel of the bottom row. Visual inspection shows minimal extraneous electrical noise with no representation of the stimulus formant structures, supporting authenticity of the evoked ECochG_{diff} responses.

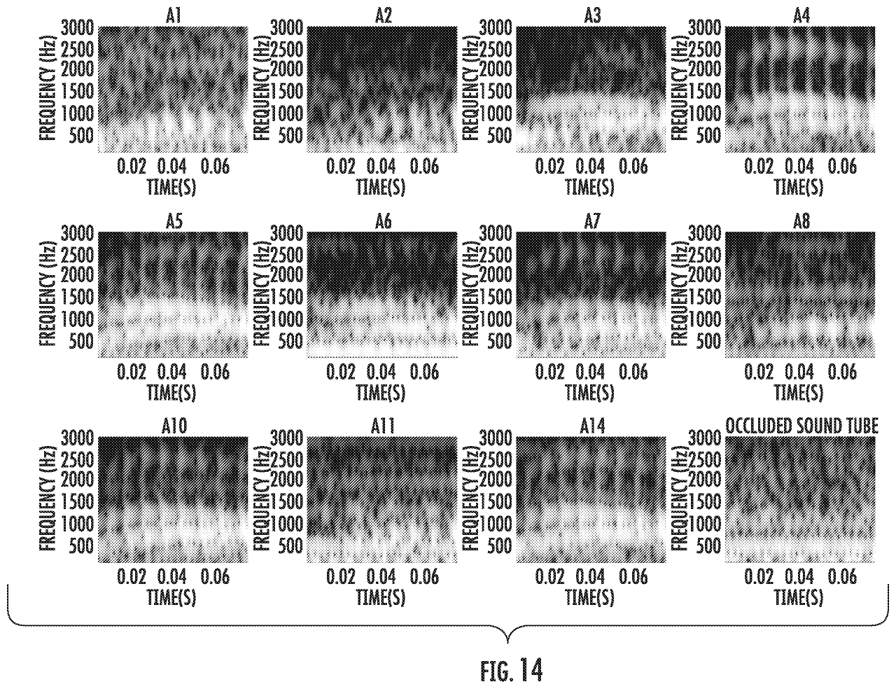

FIG. 14 displays the spectrograms for responses evoked by the /ba/ stimulus along with the averaged results of the occluded sound tube trials. Due to surgical timing constraints, A9, A12, and A13 did not have a /ba/ trial completed and were thus excluded from this analysis, thus 11 participants were included. Using the same approach as with the /da/responses, each formant structure was measured in the same manner to determine formant representation in the response. Eight participants had full F₁ representation while participants A1, A2 and A11 only exhibited partial representation of F₁. Six participants had full representation of both F₁ and F₂, of which four (A4, A5, A10, A14) also had F₃ present, while one had partial F₃ (A7) and one (A3) had no measurable F₃ response. Finally, two participants (A6, A8) had full F₁ and only partial F₂ representation in their ECochG_{diff} response. The final panel of the bottom row displays the average occluded sound tube trial for the /ba/ stimulus.

Peripheral encoding of phonemic structure: Residual hearing and speech recognition. The structural similarity index (SSIM), a mathematical calculation that evaluates structure, luminance and contrast between two images to determine their overall similarity, was used for comparison of the stimulus spectrogram with the ECochG_{diff} spectrogram. This comparison revealed that spectrograms with greater formant content had the largest SSIM values. Each participant's index value can be found in FIG. 12. The SSIM ranged from 0.18 to 0.58 (mean: 0.38, SD: 0.12) for the /da/ and 0.21 to 0.62 (mean: 0.37, SD: 0.17) for the /ba/. Of note, the CI participant with ANSD exhibited the highest SSIM values of the study group, which is not surprising as this condition is thought to result in normal hair cell function but poor or absent neural function. However, this finding suggested that better cochlear function is important for achieving higher SSIM values, thus similar values in the case of a high frequency SNHL are expected (e.g. >3 kHz).

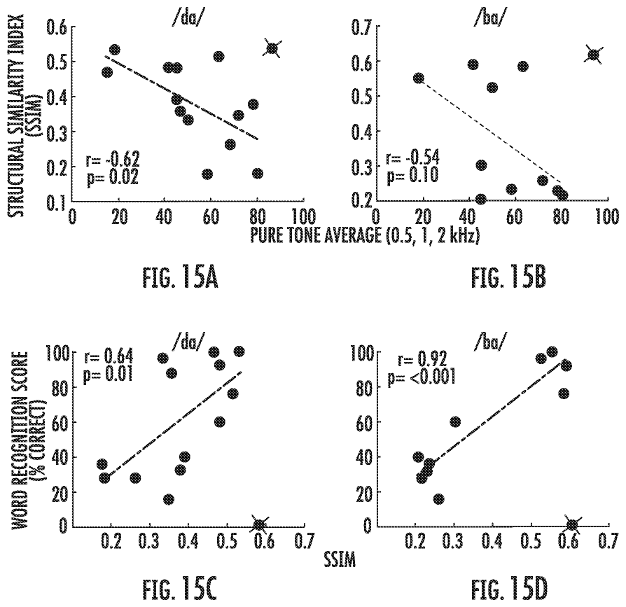

To determine the influence of residual hearing on the SSIM, the pre-operative PTA was used in a Pearson product-moment correlation with the SSIM for both stimuli (FIG. 15A for /da/ and FIG. 15B for /ba/). A significant negative correlation was found for /da/(n=13, r=−0.62, p=0.02) and a similar trend was found for /ba/ (n=10, r=−0.54, p=0.10) however this did not reach significance. This suggested that SSIM value was related to the amount of residual hearing as measured by the audiogram. Specifically, higher SSIM values were associated with better hearing and decreased in value as hearing worsened. Note, due to the nature of the hearing loss in ANSD, participant A4 was not included in these analyses with traditional SNHL participants as ANSD is known to result in neural dysfunction (e.g. temporal disruptions) leading to worse than expected WRS despite near normal cochlear function. However, the data for this subject are plotted in FIGS. 15A-15B (dot marked with “X”) to help demonstrate the strength of the SSIM when considering traditional SNHL.

Furthermore, to determine the relevance of the formant structure contained in the ECochG_{diff} response to auditory processing when evaluated using the SSIM, Pearson product-moment correlations were run between SSIM values and the behavioral performance score on the pre-operative speech perception task (e.g. WRS-%). This correlation was chosen as formant representation in the ECochG_{diff} response is expected to reflect a participant's access to spectral components of the input signal that would be important for speech perception. Indeed, the SSIM was found to positively correlate with WRS for both stimuli (/da/: n=13, r=0.64, p=0.01; /ba/: n=10, r=0.92, p<0.001) (FIG. 15C for /da/ and FIG. 15D for /ba/). As mentioned above A4 was excluded from correlations but plotted for illustrative purposes in FIGS. 15C-15D (dot marked with “X”). Overall, participants with the most residual preoperative hearing typically had higher SSIM values which correlated to the participant's word recognition capabilities.

Discussion

The example described herein demonstrates the ability to use the acoustically evoked ECochG response of the inner ear as a microphone source for representing speech properties of the input sound in a group of participants with SNHL. Participants with the greatest amount of pre-operative residual hearing (e.g. mild-to-moderate) exhibited the best frequency representation of the group to both stimuli (highest SSIM values). When considering those participants with hearing thresholds in the severe-to-profound range, most participants exhibited all of the F₁ structure and often a portion of the F₂ component as well. The proportion of formant representation in the ECochG_{diff} response (as measured by the SSIM) was significantly related to speech recognition capabilities.

Hearing status and signal representation. Typically, SNHL involves a process whereby sensory cells (outer and inner hair cells) of the cochlea are damaged and subsequently missing, leaving few sensory receptors to detect and carry out mechano-transduction and neural activation [26]. Thus, using sensory cells as an internal microphone is limited to the extent of remaining hair cell presence in the inner ear. However, previous extra and intracochlear ECochG recordings have been carried out by numerous authors in recent years in instances of severe-to-profound SNHL of which many report their ability to record ECochG activity that is thought to predominantly represent the CM [4, 6, 7, 27, 28, 29, 30]. The challenge then becomes how well the residual sensory cells can represent the incoming speech signal, and what proportion of the acoustic properties (e.g., formant structure) is necessary to be preserved for computer algorithms to accurately identify and differentiate between speech phonemes so that the appropriate signal can be delivered to the stimulating electrode array. It is demonstrated herein that despite extensive degrees of hearing loss, formant structure can be maintained to varying degrees often with at least F₁ preserved. Thus, at a minimum, it appears that simple sound detection (signal on/off) is feasible but higher signal identification (e.g., speech recognition) may be a greater challenge. For optimal results, applications of this technology can be used for CI recipients who have significant residual hearing following CI surgery, as those recipients would be most likely to maintain high-fidelity speech signals from CM responses. Additionally, while around-the-clock use of this technology may not be superior to traditional microphones in terms of speech recognition, this technique provides recipients with the option to remove the external speech processor while not completely sacrificing sound awareness.

Implications for using the biological ECochG as a microphone. While technology for development of fully-implantable CIs has been of growing interest, this study is the first report of a technique that uses a biological response as the microphone. Other techniques have been focused on using more traditional mechanical microphones such as the electret microphone [9]. Yip et al. described a proof-of-concept for a fully implantable approach using a piezoelectric middle-ear sensor in a human cadaver ear whereby the sensor output obtained from the middle ear chain is used as the sound source [10]. However, due to stability issues of placement on the middle ear ossicles, carrying this out in-vivo is a challenging prospect. Additionally, Zhao and colleagues were able to demonstrate the feasibility of designing and using an intracochlear location of a piezoelectric transducer (micro-electro-mechanical systems xylophone) in a guinea pig model [31]. Here there is a probe that courses within the cochlea and is composed of a xylophone-like structure that is designed to resonate at different frequencies in attempts to mimic the fluid dynamics of the inner ear/basilar membrane. However, the practical aspects of an additional intracochlear structure besides the electrode would need to be addressed.

One advantage of the implementations described herein is that no additional microphones would be necessary. That is, electrode arrays of CIs have several electrode contacts, and such contacts can be used to record early auditory potential such as CM. Previous work has demonstrated the feasibility of recording acoustically evoked responses from the electrode array in implanted ears [4, 5, 7]. Since these studies have shown that the maximal amplitude of the ECochG response is often found at the apical reaches of the electrode array, designating this electrode location as a constant ECochG microphone while leaving the remaining electrodes of the array to electrically stimulate the auditory nerve would not require any alteration to the normal CI surgical process or CI design.

Peripheral encoding of phonemes—Importance for speech understanding. The current assessment of signal representation used the SSIM, which is often employed in the visual sciences to compare images (reference and target). The rationale for utilizing this approach was that a single metric that could quantify overall fidelity/structure over time of the evoked response compared to the input acoustic signal can be used. Its use here yielded interesting clinical relevance. First, the amount of residual hearing, as measured by preoperative audiometry, was correlated with the SSIM. This was true for the /da/ responses and while this same trend existed for responses to /ba/, this correlation did not reach statistical significance. The smaller number of subjects available for the /ba/ correlation likely impacted this outcome. Regardless, these findings suggested that SSIM value was related to the amount of residual hearing of the participant.

Secondly, the amount of formant structure of the stimulus signal that was represented in the ECochG_{diff} response, as measured by the SSIM, strongly correlated to the participant's perceptual ability to understand speech as measured by a monosyllabic word list (NU-6). This is somewhat intuitive since SNHL is thought to result in a reduced number of hair cells and a subsequent broadening of auditory filters of the cochlea and thus reduced audibility and frequency resolution [32, 33, 34]. However, the phoneme-evoked response helps demonstrate the importance of audibility and frequency selectivity by the ear at the peripheral level and its relation to speech recognition. That is, the spectral analyses of the /da/ and /ba/evoked responses covered nearly % of the speech spectrum (bandwidth ranging from ^{˜}100 Hz to ^{˜}2500 Hz). Therefore, in the event that there were sensory hair cells in this frequency range (through ^{˜}2500 Hz) remaining that were able to accurately encode all three formants, it is expected that this spectral reach would be similar across other phonemes. This finding is attributed to similar mechanisms which underlie the speech intelligibility index (SII) [35, 36]. The basis of the SII is that the greater the residual hearing remaining to encode frequencies across the speech spectrum, the better the WRS, as long as the sound is presented at an audible level. At a loud level, WRS is predicted by the proportion of spectral encoding across most of the speech frequency bands as measured in the phoneme evoked ECochG_{diff} response. Thus, the greater proportion of the speech spectrum that is available to the participant, the better the ability to recognize speech.

As described above, this disclosure contemplates using an intracochlear electrode array to record the early auditory potentials such as CM, ideally with an apical location. For proof-of-concept, the study presented herein as an example uses an extracochlear recording location to explore the concept of a biological microphone. Previous studies have shown that when recording ECochG intracochlearly, the response can be as much as three times larger than when recording at an extracochlear location such as the RW [37]. Hence, this disclosure contemplates improved signal representation is expected when using the devices and methods describe above, for example, those described with respect to FIGS. 1-3. Note, when the ECochG_{diff} responses were reconstructed as audio files and played audibly, many of the responses were intelligible.

Conclusion

The feasibility of utilizing ECochG as a microphone in ears with varying severities of hearing loss has been demonstrated. Overall the ECochG_{diff} response exhibited modest replicability of the stimulus spectrum when residual hearing was in the mild-to-moderate range and expectedly decreased in replicability as hearing loss worsened. The similarity between the ECochG response and the stimulus (as measured by the SSIM) significantly correlating with WRS signified the importance of peripheral encoding to speech perception capability.

Methods

This study included 14 participants (13 adults [218 years] and one pediatric) undergoing various otologic/neurotologic procedures. The average age at the time of testing ranged from 13-76 years (mean 50.6 yrs, SD: 20.1 yrs).

Audiometry. As part of the standard clinical protocol at the study institution, all participants underwent a comprehensive audiometric evaluation by a licensed audiologist using a modified Hughson-Westlake procedure [38] prior to surgery. Speech recognition ability was evaluated using the Northwestern University Auditory Test No. 6 (NU-6) [39], a monosyllabic word test with a consonant-nucleus-consonant construction, presented at suprathreshold levels. Audiometric thresholds, PTA, and WRS (% correct) were obtained via chart review.

Acoustic stimuli. Target stimuli for electrophysiological testing were two synthesized (Klatt software—sold under the name SenSyn, by Sensimetrics Corporation of Malden, Mass.) consonant vowel stop bursts (48 kHz sampling rate), a 40 ms /da/ and an 80 ms /ba/, presented in alternating polarity (rarefaction/condensation). Each stimulus phase was presented for 250 repetitions for a total of 500 repetitions. These stimuli were chosen due to their established use in previous studies using complex auditory brainstem responses [40, 41, 42, 43]. Both stimuli were composed of dynamic aspects (frequency-varying). The /da/contained initial aharmonic energy components and broadband frication which is immediately followed by a spectrally dynamic formant transition to the vowel which dominates approximately % of the signal [43]. The spectrum of the /da/ consisted of a rising fundamental (F₀ [103-125 Hz]) with three formants (F₁, F₂, F₃) which vary over time from 220 to 720 Hz (F₁), 1700 to 1240 Hz (F₂), and 2580 to 2500 (F₃) over the last 30 ms of the signal. The spectrum of the /ba/ was composed of an F₀ at 120 Hz and three formants varying over time: F₁ (400 Hz-750 Hz), F₂ (1000 Hz-1200 Hz), and F₃ (2000-2500 kHz). FIGS. 8A-8F portray both stimuli in their time domains and their corresponding spectral domains. Stimulation levels were calibrated in units of dB peSPL using a 1 inch 2 cc coupler routed to a sound level meter. The sound meter used was a sound meter sold under the name SYSTEM 824 by Larson Davis of Depew, N.Y. The /da/ stimulus was presented at 108 dB peSPL while the /ba/ was presented at 89 dB peSPL. The difference in intensity was due to the interest in assessing how the ECochG response could represent multiple phonemes as well as to assess degradation caused by lower intensity levels. However, due to time constraints of performing the electrophysiological recordings during surgery, there was limited time available for data acquisition, thus two stimuli with different intensities for establishing proof-of-concept were chosen arbitrarily.

Surgical and electrocochleography recording set-up. ECochG recordings were obtained for all participants intraoperatively at the time of surgical intervention. Intraoperatively, a mastoidectomy was performed followed by a facial recess approach for all procedures (endolymphatic sac decompression and shunt [ELS], labyrinthectomy, and CI). Prior to endolymphatic sac opening (during ELS), labyrinthectomy drilling, or prior to RW opening/electrode insertion (CI surgery), a monopolar probe (Kartush raspatory probe, Plainsboro, N.J.) was positioned at the RW niche. The RW was always intact for the ECochG recordings and prior to any surgical intervention to the cochlea or vestibular structures. The evoked signal was recorded differentially from the RW probe to a reference electrode placed at the contralateral mastoid (Mc) and a ground (Neuroline 720, Ambu Inc, Ballerup, Denmark) placed at the forehead (Fz). Stimulus delivery and recording of electrophysiological responses were controlled using a Bio-logic Navigator Pro (Natus Medical Inc., San Carlos, Calif.) evoked potential system. Stimuli were delivered through a transducer (ER-3, Etymõtic Research, Elk Grove Village, Ill.) connected to a sound tube to a foam insert earphone placed in the external auditory canal. The high-pass filter was set at 70 Hz and low-pass was at 3000 Hz. Due to the recording epoch of the evoked potential equipment being fixed at 1024 points and different stimuli durations (/da/: 40 ms; /ba/: 80 ms), each /da/ trial was sampled at 16 kHz and each /ba/ trial was sampled at 12 kHz. Signals were amplified at 50,000× with artifact rejection level set at 47.5 μV. Each trial was typically followed with an occluded sound tube run (control trial) where a hemostat was placed across the sound tube blocking acoustic delivery to the ear canal, visually allowing for detection of electromagnetic contamination.

Electrophysiological analysis. ECochG results were processed off-line and analyzed using a program sold under the trademark MATLAB (R2019a) by MATHWORKS CORP. of Natick, Mass. with custom software procedures. As an objective was to evaluate the CM's representation of the speech-like stimulus signal, the condensation and rarefaction traces were extracted and used to calculate a difference curve (condensation−rarefaction=ECochG_{diff}). This calculated waveform, while not perfect at eliminating the neural portion, helped emphasize the CM response and stimulus formant structure while minimizing neural contributions from the onset CAP [7, 43, 44, 45]. After calculating the ECochG_{diff} curve, maximal amplitude defined as base-to-peak amplitude (Mv) of the non-normalized EcochG_{diff} response (time domain) measured as the point of the EcochG_{diff} response after stimulus onset that produced the maximal amplitude deflection was calculated for each participant. Subsequently, each EcochG_{diff} response was then normalized to its peak amplitude.

Stimulus-to-response correlation (Amplitude alignment). Correlation analysis was performed to quantify how well the stimulus was represented by the EcochG_{diff} response. First, in order to align the two waveforms, the EcochG_{diff} response was up-sampled to the sampling frequency of the stimulus and then shifted in time. The time shift was found by performing cross-correlation and was the lag time or latency (ms) corresponding to the point of highest correlation between the waveforms. Cross-correlation slides the EcochG_{diff} response (which has a longer recording window than the stimulus duration) along the x-axis of the stimulus (time domain) and calculates the integral of the stimulus and EcochG's product at each sampled point [46]. The point at which this calculation is maximized becomes the point of alignment between the stimulus and ECochG_{diff} response. Thus, the ECochG_{diff} response is then shifted according to the latency. After alignment of the signals, the ECochG_{diff} response was windowed from 0-40 ms (same time scale as the /da/ stimulus) or 0-80 ms (same time scale as the /ba/ stimulus). Finally, Pearson product-moment correlation (r) between the two waveforms was calculated and description of correlation strength (e.g. “moderate”) was based on Cohen's r classification system [47]. This approach established similarity between waveform morphology of ECochG_{diff} and stimulus within the time-domain. For Pearson correlations, all tests were two tailed and statistical significance was determined at the 95% confidence level.

Spectrogram and structural similarity index (SSIM). After time-domain alignment the ECochG_{diff} response was analyzed in its frequency domain using spectrogram analysis to evaluate spectro-temporal aspects (frequency variations over time). Spectrograms contained time segments composed of 240 points each that were each shaped by a Hamming window, were broad-band with a window length of 0.005 seconds (helped emphasize formant structure rather than pitch (F₀) structure), had a frequency step of 10 Hz, were displayed with a view range of 70 Hz-3000 Hz (same as ECochG filter settings), and were then gray-scaled (intensity range of 0-1). Frequency content for each portion of the spectrogram was calculated using FFTs with zero padding on each windowed time segment. To descriptively classify whether full or partial formant structure was present, the noise floor three bins above and below the boundary of the formant frequency of interest for three regions along the entire formant (beginning, middle, end) was estimated and the results were 18, 25, and 35 ms for the /da/ and 12, 40, and 68 ms for the /ba/. If all three regions were each three standard deviations above the noise floor (measured from three bins above and below the region of interest), then full formant representation was considered preserved. If only one or two of these regions were significant, then the formant structure was classified as partially present. Additionally, an occluded sound trial was conducted to confirm authenticity of the ECochG_{diff} response whereby the response was visually inspected for evidence of electromagnetic contamination (speaker artifact resembling the stimulus signal) of which no trial was found to contain this artifact.

Furthermore, as an objective was to determine how well the biological ECochG response could serve as a microphone, it was desirable to compare the frequency spectrum of the ECochG_{diff} response to that of the complex stimulus signal. SSIM was chosen to evaluate the spectra between the ECochG_{diff} response and stimulus. As formant structure is critical for differentiation of phonemes (/da/ vs /ba/), a technique that is sensitive to structural preservation (i.e. quantity and quality) can be used. The SSIM is a technique designed to evaluate two images (e.g. spectrograms), a reference (e.g. stimulus) and an image of interest (e.g. ECochG_{diff}), and determine the overall similarity (distortion/error) of the two images by calculating a single overall similarity value (index) [48, 49]. SSIM indices range from −1 to 1 where 1 indicates complete structural similarity (only achievable when two images are identical), 0 represents no similarity, and −1 being an exact opposite. Its value is the output of three computations between the signal spectrogram and ECochG_{diff} spectrogram: (1) linear correlation of the two signals, (2) mean luminance and (3) mean contrast. This index value was then used in separate correlations (Pearson) with PTA and WRS to evaluate clinical relevance of formant structure representation in the ECochG_{diff} response. Linear regression (least-squares) was then used to determine a line of best fit for each correlation. All statistical tests were two-tailed with significance determined at the 95% confidence level.

### REFERENCES

[1] Stevens, G. et al. Global and regional hearing impairment prevalence: an analysis of 42 studies in 29 countries. Eur. J. Public. Health 23, 146-152 (2013).
[2] Gantz, B. J. & Turner, C. W. Combining acoustic and electrical hearing. Laryngoscope 113, 1726-1730 (2003).
[3] Turner, C. W., Reiss, L. A. J. & Gantz, B. J. Combined acoustic and electric hearing: Preserving residual acoustic hearing. Hearing Res. 242, 164-171(2008).
[4] Campbell, L., Kaicer, A., Briggs, R. & O'Leary, S. Cochlear response telemetry: intracochlear electrocochleography via cochlear implant neural response telemetry pilot study results. Otol. Neurotol. 36, 399-405 (2015).
[5] Harris, M. S. et al. Patterns Seen During Electrode Insertion Using Intracochlear Electrocochleography Obtained Directly Through a Cochlear Implant. Otol. Neurotol. 38, 1415-1420 (2017).
[6] Harris, M. S. et al. Real-Time Intracochlear Electrocochleography Obtained Directly Through a Cochlear Implant. Otol. Neurotol. 38, e107-e113 (2017).
[7] Koka, K., Saoji, A. A. & Litvak, L. M. Electrocochleography in Cochlear Implant Recipients With Residual Hearing: Comparison With Audiometric Thresholds. Ear Hear. 38, e161-e167 (2017).
[8] Zeng, F. G., Rebscher, S., Harrison, W., Sun, X. & Feng, H. Cochlear implants: system design, integration, and evaluation. IEEE Rev. Biomed. Eng. 1, 115-142 (2008).
[9] Briggs, R. J. et al. Initial clinical experience with a totally implantable cochlear implant research device. Otol. Neurotol. 29, 114-119 (2008).
[10] Yip, M., Jin, R., Nakajima, H. H., Stankovic, K. M. & Chandrakasan, A. P. A Fully-Implantable Cochlear Implant SoC with Piezoelectric Middle-Ear Sensor and Energy-Efficient Stimulation in 0.18 mu m HVCMOS. Isscc Dig. Tech. Pap. I 57, 312-+ (2014).
[11] Dallos, P., Schoeny, Z. G. & Cheatham, M. A. Cochlear summating potentials. Descriptive aspects. Acta Otolaryngol. Suppl. 302, 1-46 (1972).
[12] Davis, H., Deatherage, B. H., Eldredge, D. H. & Smith, C. A. Summating potentials of the cochlea. Am. J. Physiol. 195, 251-261 (1958).
[13] Goldstein, M. H. & Kiang, N. Y. S. Synchrony of Neural Activity in Electric Responses Evoked by Transient Acoustic Stimuli. J. Acoustical Soc. Am. 30, 107-114 (1958).
[14] Chertoff, M., Lichtenhan, J. & Willis, M. Click- and chirp-evoked human compound action potentials. J. Acoustical Soc. Am. 127, 2992-2996 (2010).
[15] Dallos, P. The Auditory Periphery Biophysics and Physiology. (Academic Press, Inc, 1973).
[16] Wever, E. G. & Bray, C. Action currents in the auditory nerve in response to acoustic stimulation. Proc. Nat. Acad. Sci., USA 16, 344-350 (1930).
[17] Dallos, P. & Cheatham, M. A. Production of cochlear potentials by inner and outer hair cells. J. Acoust. Soc. Am. 60, 510-512 (1976).
[18] Fant, G. Acoustic Theory of Speech Production. (Mouton & Co., 1960).
[19] Peterson, G. E. & Barney, H. L. Control Methods Used in a Study of the Vowels. J. Acoustical Soc. Am. 24, 175-184 (1952).
[20] Leek, M. R. & Summers, V. Auditory filter shapes of normal-hearing and hearing-impaired listeners in continuous broadband noise. J. Acoust. Soc. Am. 94, 3127-3137 (1993).
[21] Pick, G. In Psychophysics and Physiology of Hearing (eds E. F. Evans & J. P. Wilson) (Academic, 1977).
[22] Nabelek, A. K., Czyzewski, Z., Krishnan, L. A. & Krishnan, L. A. The Influence of Talker Differences on Vowel Identification by Normal-Hearing and Hearing-Impaired Listeners. J. Acoustical Soc. Am. 92, 1228-1246 (1992).
[23] Owens, E., Talbott, C. B. & Schubert, E. D. Vowel Discrimination of Hearing-Impaired Listeners. J. Speech Hearing Res. 11, 648-&(1968).
[24] Pickett, J. M., Martin, E. S., Brand Smith, S., Daniel, Z. & Willis, D. In Speech Communication Ability and Profound Deafness (ed Fant, G) 119-134 (Alexander Graham Bell Association for the Deaf, 1970).
[25] Van Tasell, D. J., Fabry, D. A. & Thibodeau, L. M. Vowel identification and vowel masking patterns of hearing-impaired subjects. J. Acoust. Soc. Am. 81, 1586-1597 (1987).
[26] Pickles, J. O., Comis, S. D. & Osborne, M. P. Cross-links between stereocilia in the guinea pig organ of Corti, and their possible relation to sensory transduction. Hear. Res. 15, 103-112 (1984).
[27] Choudhury, B. et al. Intraoperative round window recordings to acoustic stimuli from cochlear implant patients. Otol. Neurotol. 33, 1507-1515 (2012).
[28] Fitzpatrick, D. C. et al. Round window electrocochleography just before cochlear implantation: relationship to word recognition outcomes in adults. Otol. Neurotol. 35, 64-71(2014).
[29] Riggs, W. J. et al. Intraoperative Electrocochleographic Characteristics of Auditory Neuropathy Spectrum Disorder in Cochlear Implant Subjects. Front. Neurosci. 11, 416 (2017).
[30] Fontenot, T. E. et al. Residual Cochlear Function in Adults and Children Receiving Cochlear Implants: Correlations With Speech Perception Outcomes. Ear Hearing 40, 577-591 (2019).
[31] Zhao, C. M. et al. Voltage readout from a piezoelectric intracochlear acoustic transducer implanted in a living guinea pig. Scientific Reports 9 (2019).
[32] Tyler, R. S., Hall, J. W., Glasberg, B. R., Moore, B. C. J. & Patterson, R. D. Auditory Filter Asymmetry in the Hearing-Impaired. J. Acoustical Soc. Am. 76, 1363-1368 (1984).
[33] Glasberg, B. R. & Moore, B. C. J. Auditory Filter Shapes in Subjects with Unilateral and Bilateral Cochlear Impairments. J. Acoustical Soc. Am. 79, 1020-1033 (1986).
[34] Leek, M. R. & Summers, V. Auditory Filter Shapes of Normal-Hearing and Hearing-Impaired Listeners in Continuous Broad-Band Noise. J. Acoustical Soc. Am. 94, 3127-3137 (1993).
[35] Kryter, K. D. Methods for Calculation and Use of Articulation Index. J. Acoustical Soc. Am. 34, 1689-& (1962).
[36] ANSI. Vol. S3.5 (R2007) (Acoustical Society of America, New York, 1997).
[37] Calloway, N. H. et al. Intracochlear electrocochleography during cochlear implantation. Otol. Neurotol. 35, 1451-1457 (2014).
[38] Carhart, R. & Jerger, J. F. Preferred method for clinical determination of pure-tone thresholds. J. Speech Hear. Disord. 24, 330-345 (1959).
[39] Tillman, T. W. & Carhart, R. An expanded test for speech discrimination utilizing CNC monosyllabic words. Northwestern University Auditory Test No. 6. SAM-TR-66-55. Tech Rep SAM-TR, 1-12 (1966).
[40] Russo, N., Nicol, T., Musacchia, G. & Kraus, N. Brainstem responses to speech syllables. Clin. Neurophysiol. 115, 2021-2030 (2004).
[41] Akhoun, I. et al. The temporal relationship between speech auditory brainstem responses and the acoustic pattern of the phoneme vertical bar ba vertical bar in normal-hearing adults. Clin. Neurophysiol. 119, 922-933 (2008).
[42] Song, J. H., Nicol, T. & Kraus, N. Test-retest reliability of the speech-evoked auditory brainstem response. Clin. Neurophysiol. 122,346-355 (2011).
[43] Skoe, E. & Kraus, N. Auditory brainstem response to complex sounds: a tutorial. Ear Hearing 31, 302-324 (2010).
[44] Aiken, S. J. & Picton, T. W. Human cortical responses to the speech envelope. Ear Hearing 29, 139-157 (2008).
[45] Heinz, M. G. & Swaminathan, J. Quantifying Envelope and Fine-Structure Coding in Auditory Nerve Responses to Chimaeric Speech. Jaro-J Assoc. Res. Oto 10, 407-423 (2009).
[46] Derrick, T. R. & Thomas, J. M. In Innovative analysis of human movement (ed. Stergiou, N.) 189-205 (Human Kinetics Publishers, 2004).
[47] Cohen, L. H. In Life Events and Psychological Functioning: Theoretical and Methodological Issues (ed. Cohen, L. H) 11-30 (Sage, 1988).
[48] Wang, Z. & Bovik, A. C. A universal image quality index. Ieee Signal. Proc. Let. 9, 81-84 (2002).
[49] Wang, Z., Bovik, A. C., Sheikh, H. R. & Simoncelli, E. P. Image quality assessment: From error visibility to structural similarity. IEEE T Image Process. 13, 600-612 (2004).

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. An auditory prosthetic device, comprising:
an electrode array that is configured for insertion into at least a portion of a subject's inner ear; and
a receiver-stimulator operably coupled to the electrode array, wherein the electrode array is configured for electrical recording and stimulation.

2. The auditory prosthetic device of claim 1, wherein the receiver-stimulator is configured to:
receive an early auditory potential recorded by the electrode array, wherein the early auditory potential is recorded using the electrode array, and wherein the early auditory potential comprises a cochlear microphonic;
process the early auditory potential to generate a stimulation signal; and
transmit the stimulation signal to the electrode array.

3. The auditory prosthetic device of claim 2, wherein the stimulation signal is applied within the subject's cochlea using the electrode array.

4. The auditory prosthetic device of claim 1, wherein the receiver-stimulator comprises a digital signal processor (DSP), and wherein the DSP is configured to process the early auditory potential to generate the stimulation signal.

5. The auditory prosthetic device of claim 2, wherein processing the early auditory potential to generate the stimulation signal comprises detecting and removing a stimulus artifact.

6. The auditory prosthetic device of claim 5, wherein the stimulus artifact is detected and removed using at least one of a template matching technique, a linear interpolation technique, or low pass filtering.

7. The auditory prosthetic device of claim 1, wherein the electrode array comprises a plurality of electrodes.

8. The auditory prosthetic device of claim 7, wherein the early auditory potential is recorded at one or more of the electrodes of the electrode array.

9. The auditory prosthetic device of claim 7, wherein the early auditory potential is recorded at each of the electrodes of the electrode array.

10. The auditory prosthetic device of claim 7, wherein the electrodes of the electrode array are arranged to correspond to different tonotopic locations of the subject's cochlea.

11. The auditory prosthetic device of claim 2, wherein the early auditory potential further comprises at least one of a compound action potential (CAP), a summating potential (SP), or an auditory nerve neurophonic (ANN).

12. The auditory prosthetic device of claim 1, wherein the auditory prosthetic device is a cochlear implant.

13. The auditory prosthetic device of claim 1, wherein the auditory prosthetic device is an implantable device.

14. The auditory prosthetic device of 13, wherein the auditory prosthetic device is a semi-implantable device.

15. A method for using early auditory potentials in an auditory prosthetic device, comprising:
recording, using an electrode array, an early auditory potential, wherein the early auditory potential comprises a cochlear microphonic;
processing, using a digital signal processor (DSP), the early auditory potential to generate a stimulation signal; and
transmitting the stimulation signal to the electrode array.

16. The method of claim 15, further comprising applying, using the electrode array, the stimulation signal within the subject's cochlea.

17. The method of claim 16, wherein the electrode array is used to both record the early auditory potential and apply the stimulation signal.

18. The method of claim 15, wherein processing, using the DSP, the early auditory potential to generate the stimulation signal comprises detecting and removing a stimulus artifact.

19. The method of claim 15, wherein the early auditory potential further comprises at least one of a compound action potential (CAP), a summating potential (SP), or an auditory nerve neurophonic (ANN).

20. The method of claim 15, wherein the electrode array is inserted into at least a portion of the subject's inner ear.
